(19) United States
(12) Patent Application Publication
(10) Pub. No.: US 2011/0060175 A1
(43) Pub. Date: Mar. 10, 2011

(54) METHOD FOR MANUFACTURING COMPOUNDS HAVING AN ADAMANTANE STRUCTURE

(75) Inventors: Ikuko Takahashi, Chiba (JP); Akio Kojima, Chiba (JP)

(73) Assignee: IDEMITSU KOSAN CO., LTD, Tokyo (JP)

(21) Appl. No.: 12/991,246

(22) PCT Filed: Apr. 23, 2009
(86) PCT No.: PCT/JP09/58062

§ 371 Date: Nov. 5, 2010

(30) Foreign Application Priority Data
May 7, 2008 (JP) 2008-121613

(51) Int. Cl. C07C 2/74

(52) U.S. Cl. 585/405; 585/404

(57) ABSTRACT

[Problem to be Solved]The object of the present invention is to provide an industrially favorable production process whereby adamantanes can be produced in a high yield using a catalyst, which is used for the isomerization reaction of a tricyclic saturated hydrocarbon compound having 10 or more carbon atoms, does not require a complicated waste liquid treatment operation, and makes high-yield production of adamantanes possible.
[Solution] A process for producing a compound having an adamantane structure, wherein one or more catalysts selected from the following (a) to (c) are used:
(a) zeolite having MWW topology;
(b) delaminated MWW-type zeolite; and
(c) interlayer-expanded MWW-type zeolite prepared by treatment with a metal compound.

### TECHNICAL FIELD

The present invention relates to a novel process for producing compounds having an adamantane structure. In more detail, the present invention relates to an industrially favorable process for producing compounds having an adamantane structure (hereinafter, may sometimes be referred to as adamantanes) in a high yield by isomerizing a tricyclic saturated hydrocarbon compound having 10 or more carbon atoms by using a specific zeolite catalyst.

### BACKGROUND ART

Adamantane has a structure in which four cyclohexane rings are bound in a cage-like structure and is a stable compound with high symmetry. Adamantanes having such an adamantane structure show unusual functions and are known to be useful as raw materials for lubricants, electronic materials such as resists and the like, or agrochemicals and drugs, and as raw materials for highly functional industrial materials. As a process for producing these adamantanes, there is generally employed a process wherein a tricyclic saturated hydrocarbon compound having 10 or more carbon atoms is isomerized. For example, adamantane is obtained by catalytically isomerizing trimethylenenorbornane (TMN) obtained by hydrogenating dicyclopentadiene (DCPD) and as the catalyst, there is industrially used aluminum chloride (see, for example, PTL 1).

Additionally, in a batch reaction, there is known a process for producing adamantanes, wherein a catalyst is used that has a metal such as platinum, rhenium, nickel, cobalt, and the like loaded by an impregnation method on cation-exchanged ultra-stable Y-type zeolite or Y-type zeolite (see, for example, NPLs 1 and 2).

There is also known a process for producing adamantanes, wherein there is used a solid catalyst having an active metal such as platinum, rhenium, nickel, cobalt, and the like loaded on cation-exchanged zeolite (see, for example, PTL 2). Also, when producing hydrocarbons having an adamantane structure, it is known to use, as the isomerization catalyst, cation-exchanged zeolite having an active metal loaded thereon, which has been treated with ammonium sulfate (see, for example, PTL 3).

Furthermore, there are known a catalyst having an active metal loaded on a solid acid, wherein the amount of alkali metals is controlled at a specified value or lower, and a process for producing adamantanes using the catalyst (see, for example, PTL 4).

However, when producing adamantanes using aluminum chloride as the catalyst, the amount of the catalyst relative to the raw material has to be increased and, moreover, because this catalyst forms complexes with heavy fractions produced as byproducts during the course of the reaction, the catalyst cannot be reused. Therefore, when this process is employed, a large amount of waste aluminum is generated, with the waste treatment causing a problem of environmental pollution. In addition, because aluminum chloride is highly corrosive, it is necessary to use expensive equipment made of corrosion-resistant material. Further, when aluminum chloride is used, there is a drawback that the adamantanes produced become discolored and, therefore, a recrystallization step and a decolorization step by using activated carbon and the like become necessary, making the after-treatment complicated.

With the catalysts disclosed in NPLs 1 and 2, the yield of adamantanes becomes relatively high but these require coexistence of hydrogen chloride; when hydrogen chloride does not coexist, the yield of adamantanes becomes lower. Because hydrogen chloride is highly corrosive, there arise problems such as necessity to use expensive anticorrosive equipment and the like.

Additionally, in the case of a production process disclosed in PTL 4 where adamantanes are produced without coexistence of hydrogen chloride in a flow reaction by using a catalyst having platinum loaded on cation-exchanged Y-type zeolite in an amount of 1 mass % or less, the selectivity for and yield of adamantanes become lower because a large amount of hydrogenolysis product is produced by a side reaction (conversion of TMN: 91.5%, selectivity for adamantane: 16.9%, and yield of adamantane: 15.5%). Further, in order to suppress deactivation of the catalyst, a condition of high-pressure hydrogen is indispensable, making it difficult to control the hydrogenolysis byproduct. Thus, this process has a drawback that improvement of selectivity for adamantane is difficult.

### [Citation List]

### [Patent Literature]

[PTL 1] Japanese Patent Laid-Open Publication No. H2-235826
[PTL 2] Japanese Patent Application Publication No. S52-2909
[PTL 3] Japanese Patent Laid-Open Publication No. S60-246333
[PTL 4] Japanese Patent Laid-Open Publication No. 2005-118718

### [Non Patent Literature]

[NPL 1] Guo Jianwei et at, PETROCEMICHAL INDUSTRY, 1998, vol. 27, No.1
[NPL 2] GAO ZI et al., CHINESE Journal of chemistry, 1994, vol. 12, No. 1

### SUMMARY OF INVENTION

### [Problem to be Solved by Invention]

Under these circumstances, an object of the present invention is to provide an industrially favorable production process whereby adamantanes can be produced in a high yield using a catalyst, which is used for the isomerization reaction of a tricyclic saturated hydrocarbon compound having 10 or more carbon atoms, does not necessitate a complicated waste liquid treatment operation, and makes high-yield production of adamantanes possible.

### [Means for Solving the Problem]

In order to attain the above object, the present inventors conducted diligent research and, as a result, found that by carrying out isomerization of a tricyclic saturated hydrocarbon compound having 10 or more carbon atoms by using a specific zeolite catalyst, adamantanes can be produced in a high yield without necessity of a complicated waste liquid treatment operation.

That is, the present invention is:

(1) a process for producing a compound having an adamantane structure, wherein one or more catalysts selected from the following (a) to (c) are used:

(a) zeolite having MWW topology;

(b) delaminated MWW-type zeolite; and

(c) interlayer-expanded MWW-type zeolite prepared by treatment with a metal compound,

(2) the production process according to (1) above, wherein the (a) zeolite having MWW topology is one selected from MCM-22, SSZ-25, ITQ-1, PSH-3, and ERB-1,
(3) the production process according to (1) above, wherein the (b) delaminated MWW-type zeolite is ITQ-2,
(4) the production process according to (1) above, wherein the (c) inter-layer expanded MWW-type zeolite prepared by treatment with a metal compound is MCM-36,
(5) the production process according to any one of (1) to (4) above, wherein production is conducted by isomerizing a tricyclic saturated hydrocarbon compound having 10 or more carbon atoms by using one or more catalysts selected from (a) to (c) according to paragraph (1) above,
(6) the production process according to (5) above, wherein the tricyclic saturated hydrocarbon compound is at least one selected from trimethylenenorbornane, dimethyltrimethylenenorbornane, perhydroaccnaphthene, and perhydrofluorene,
(7) the production process according to any of (1) to (6) above, wherein one or more catalysts selected from (a) to (c) according to paragraph (1) above is one having an active metal loaded thereon,
(8) the production process according to (7) above, wherein the active metal is at least one selected from metals belonging to Group 8 to Group 10 of the periodic table and Re,
(9) the production process according to (7) or (8) above, wherein the active metal is platinum, and
(10) the production process according to any one of (7) to (9) above, wherein the amount of the active metal loaded relative to the catalyst is 1 mass % or less.

### [Effects of Invention]

According to the present invention, there can be provided an industrially favorable production process whereby adamantanes can be produced in a high yield using a catalyst, which is used for the isomerization reaction of a tricyclic saturated hydrocarbon compound having 10 or more carbon atoms, does not necessitate a complicated waste liquid treatment operation, and makes high-yield production of adamantanes possible.

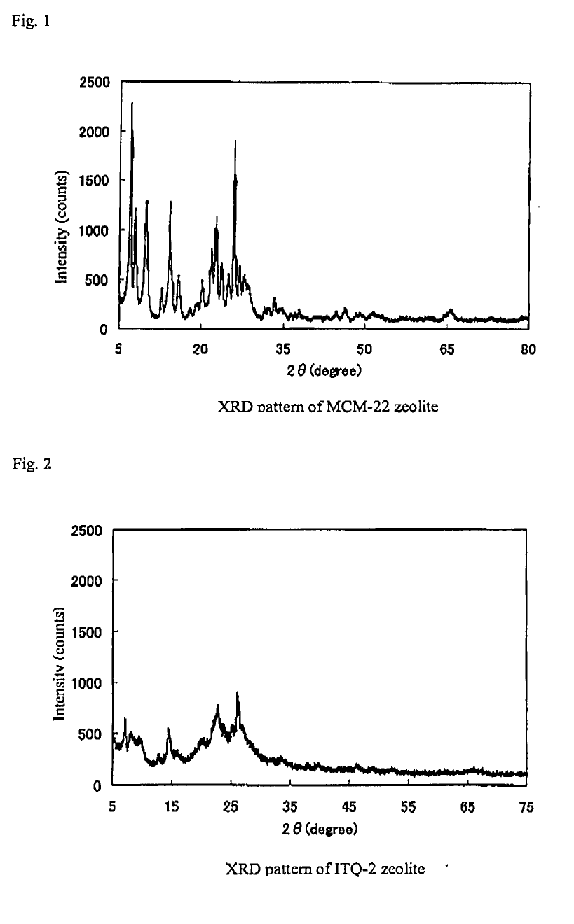

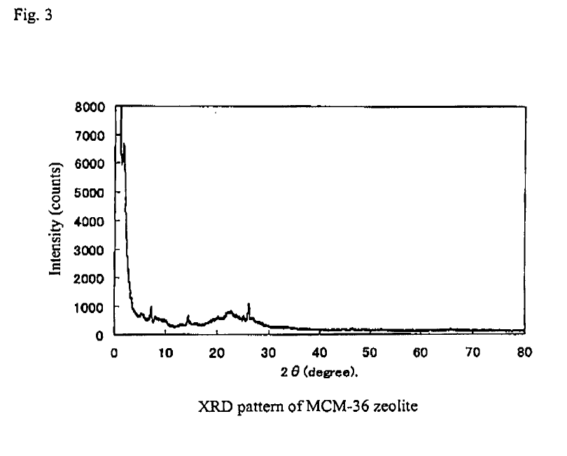

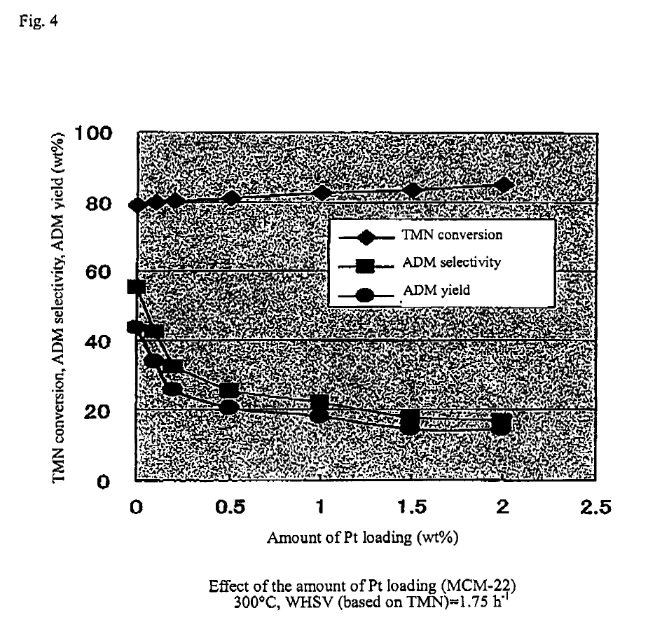

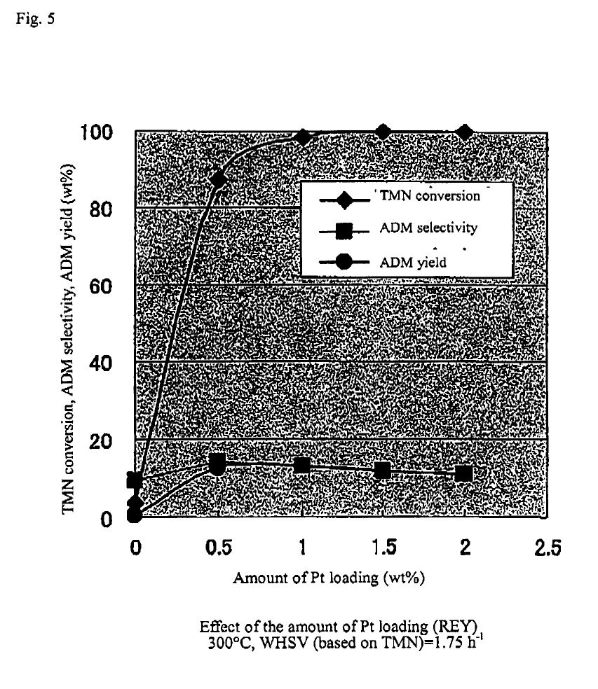

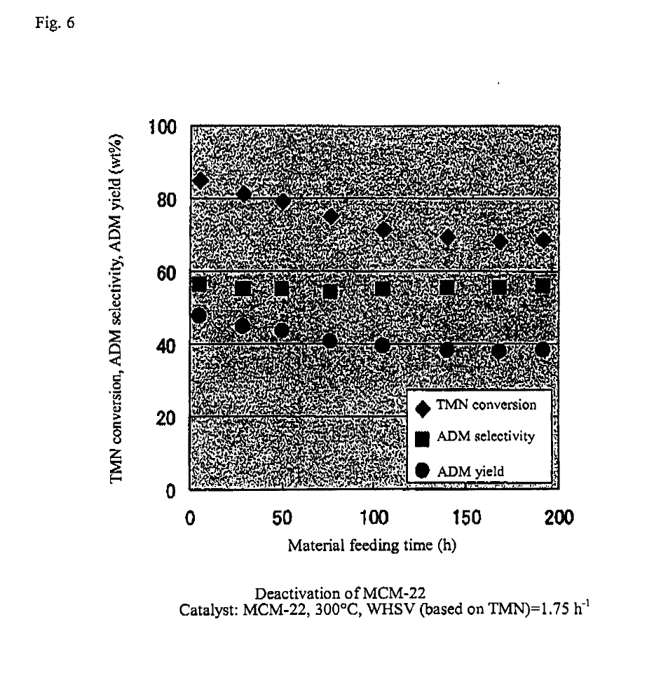

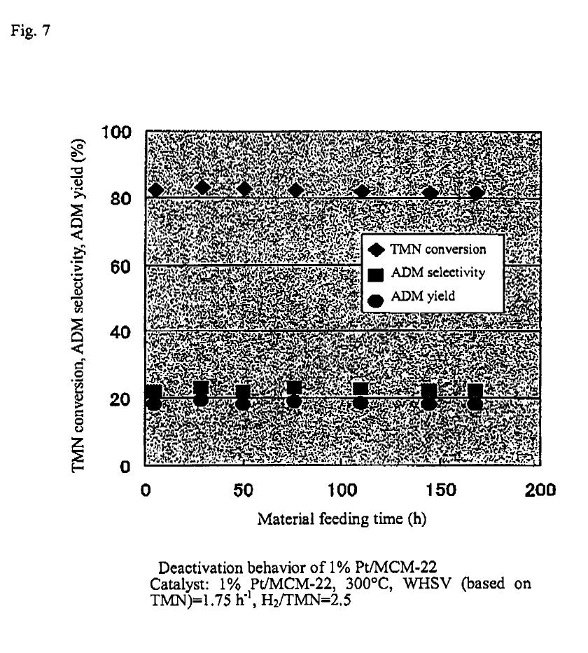

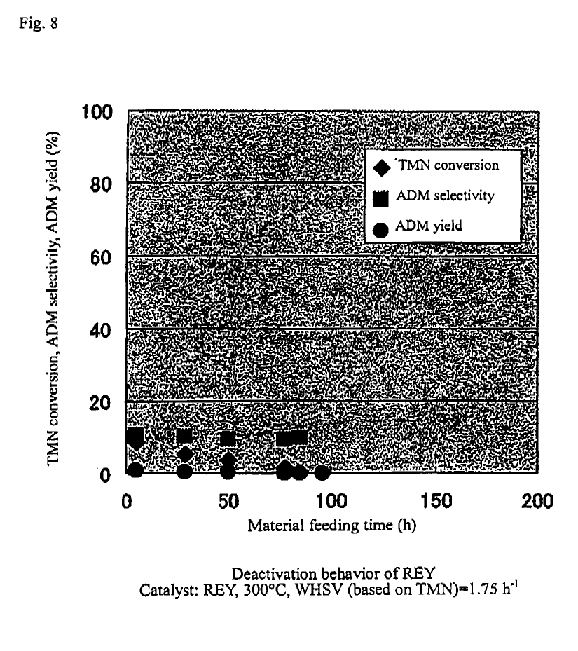

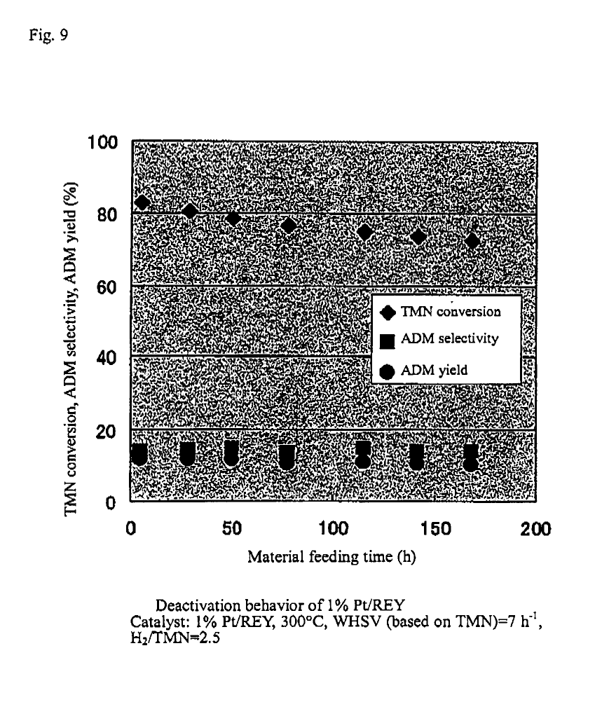

### BRIEF DESCRIPTION OF DRAWINGS

[FIG. 1] This is an X-ray diffraction pattern of MCM-22 zeolite.

[FIG. 2] This is an X-ray diffraction pattern of ITQ-2 zeolite.

[FIG. 3] This is an X-ray diffraction pattern of MCM-36zeolite.

[FIG. 4] This is a figure showing the effect of the amount of Pt loading on MCM-22 zeolite.

[FIG. 5] This is a figure showing the effect of the amount of Pt loading on REY zeolite.

[FIG. 6] This is a figure showing deactivation behavior of MCM-22 zeolite.

[FIG. 7] This is a figure showing deactivation behavior of MCM-22 zeolite having 1% Pt loading thereon.

[FIG. 8] This is a figure showing deactivation behavior of REY zeolite.

[FIG. 9] This is a figure showing deactivation behavior of REY zeolite having 1% Pt loading thereon.

### PREFERRED EMBODIMENTS

Hereinafter, the present invention will be described in more detail.

In the present invention, the catalyst used for producing adamantanes is one or more catalysts selected from (a) zeolite having MWW topology, (b) delaminated MWW-type zeolite, and (c) interlayer-expanded MWW-type zeolite prepared by treatment with a metal compound. As the (a) zeolite having MWW topology, preferable are MCM-22 zeolite, SSZ-25 zeolite, ITQ-1 zeolite, PSH-3 zeolite, and ERB-1 zeolite. Or, as the (b) delaminated MWW-type zeolite, preferable is ITQ-2 zeolite and, as the (c) interlayer-expanded MWW-type zeolite by treatment with metal compounds, preferable is MCM-36 zeolite. Among these, more preferable are MCM-22 zeolite, ITQ-2 zeolite, and MCM-36 zeolite; and most preferable is MCM-22 zeolite.

The (a) zeolite having MWW topology may, for example, be prepared by a process described in the Example of U.S. Pat. No. 4,954,325 (MCM-22); the (b) delaminated MWW-type zeolite may, for example, be obtained by a process described in WO 97/17290 (ITQ-2); and the (c) interlayer-expanded MWW-type zeolite prepared by treatment with metal compounds may, for example, be obtained by a method described in WO 92/11934 (MCM-36).

Nomenclature of zeolite materials is determined by the Structure Commission of the International Zeolite Association (IZA-SC). To the Structure Commission is given the authority by the IUPAC to assign framework type codes to all zeolites having confirmed and unique framework topologies. At present, the final terminology is described in the “Atlas of Zeolite Structure Types” (4th Edition, Authors: W. M. Meier, D. H. Olson, Ch. Baerlocher) and periodically revised records can be accessed through the following website: www.iza-sc.ethz.ch/IZA-SC/AtlasHome.html. In this handbook, the topology of each zeolite type which is thought to have a novel independent structure is recorded and, at present, about 125 kinds of independent zeolite structures are listed. The zeolite materials assigned to the MWW topology by the IZA-SC are multilayered materials and contain two kinds of fine pores due to the presence of 10-membered rings and 12-membered rings. In the “Atlas of Zeolite Structure Types”, the zeolites having this same topology are classified into the following five different materials: MCM-22 zeolite, ERB-1 zeolite, ITQ-1 zeolite, PSH-3 zeolite, and SSZ-25 zeolite. The MWW zeolite is described as a material having various applications. In the description of U.S. Pat. No. 4,826,667, it is described that SSZ-25 zeolite is not only useful mainly in catalytic transformation reactions of hydrocarbons such as catalytic cracking, hydrogenolysis, hydrodewaxing, and formation reactions of olefins and aromatic compounds (for example, xylene isomerization), but also useful as an adsorbent, fillers, and a water softening agent. In U.S. Pat. No. 4,954,325, there are described 16 different applications for a material known as MCM-22.

As the tricyclic saturated hydrocarbon compounds having 10 or more carbon atoms, which are used as the raw material in the present invention, especially preferable are tricyclic saturated hydrocarbon compounds having 10 to 15 carbon atoms, wherein the C—C bond strain is relatively high. For example, there may be mentioned trimethylenenorbomane [tetrahydrodicyclopentadiene], perhydroacenaphthene, perhydrofluorene, perhydrophenalene, 1,2-cyclopentanoperhydronaphthalene, perhydroanthracene, perhydrophenanthrene, and the like. Further, there may also be mentioned alkyl substituted derivatives of these compounds, for example, 9-methylperhydroanthracene and the like as preferable compounds. Among these, trimethylenenorbornane is especially preferable.

These tricyclic saturated hydrocarbon compounds having 10 or more carbon atoms can easily be obtained by hydrogenating raw materials such as dicyclopentadiene, acenaphthene, and the like in the presence of a publicly known hydrogenation catalyst such as, for example, Raney-Ni, Pt, and the like.

From a standpoint of suppressing catalyst deactivation, an active metal may be loaded on the catalyst for producing adamantanes of the present invention. For example, preferable are rare-earth metals, alkaline earth metals, metals which belong to Group 8 to Group 10 of the periodic table, and Re. More preferable are Ru, Rh, Pd, Ir, Pt, and Re, and especially preferable is Pt. These metals may be loaded singly or in a combination of two or more kinds. The amount of the active metal to be loaded is not particularly limited but, from a viewpoint of catalytic activity, it is usually 0.0001 to 1 mass % based on the total weight of the catalyst. Within this range, adamantanes can be obtained in a high yield.

Methods for loaded an active metal include an ion exchange method and/or an impregnation method, whereby at least one of the active metals is loaded on predetermined zeolite.

In the case of the ion exchange method, an aqueous solution of a salt or a complex of the active metal is brought into contact with the predetermined zeolite so that the cation sites in this zeolite, for example, alkali metal ions, H⁺, NH₄⁺, and the like are ion-exchanged. Thereafter, the zeolite is subjected to drying treatment followed by calcination treatment to obtain the desired catalyst.

In the case of the impregnation method, after mixing the predetermined zeolite and a salt or complex of the active metal, water is distilled off according to a conventional method and, subsequently, the dried material is calcined to obtain the desired catalyst.

Temperature of the calcination treatment is suitably chosen depending on the kind of metal used in the ion exchange method, the kind of metal used in the impregnation method, and the like. The shape of the thus obtained catalyst of the present invention is not particularly limited and may be any of powder, granules, cylinders, and the like,

In the present invention, when isomerizing a tricyclic saturated hydrocarbon compound having 10 or more carbon atoms in the presence of the aforementioned catalyst, the reaction may be carried out under coexistence of a monocyclic saturated hydrocarbon compound, an aromatic compound, water and/or alcohols, and the like. Here, the monocyclic saturated hydrocarbon compounds, which may be made to coexist, include, for example, cyclopentane, cyclohexane, ethylcyclohexane, methylcyclohexane, and the like. Especially, cyclohexane, ethylcyclohexane, or a mixture of these is preferable. Also, the aromatic compounds include, for example, aromatic hydrocarbons such as benzene, toluene, xylene, naphthalene, anthracene, and the like; oxygen-containing aromatic compounds such as phenol, benzaldehyde, benzoic acid, benzyl alcohol, anisole, and the like; nitrogen-containing aromatic compounds such as aniline, nitrobenzene, and the like; halogen-containing aromatic compounds such as chlorobenzene, bromobenzene, and the like. Among these aromatic compounds, especially preferable are aromatic hydrocarbon compounds such as benzene, toluene, xylene, naphthalene, anthracene, and the like. On the other hand, the alcohols include, for example, monovalent alcohols such as methyl alcohol, ethyl alcohol, isopropyl alcohol, tert-butyl alcohol, benzyl alcohol, and the like; and polyvalent alcohols such as ethylene glycol, glycerin, and the like.

The amount of these compounds which are added and made to coexist is not particularly limited and can be selected suitably depending on various circumstances.

The reaction temperature is usually in the range of 150 to 450° C., preferably 200 to 400° C., and more preferably 250 to 350° C. Within this range, the higher the temperature, the higher the yield of adamantanes. When the temperature is too low, the conversion of the raw material becomes smaller and the yield of adamantanes is decreased. When the temperature is too high, the amount of byproducts due to a degradation reaction increases, resulting in lower selectivity for adamantanes and, thus, a lower yield of adamantanes.

The reaction is carried out under normal pressure or under increased pressure. It is preferable to conduct the reaction under increased pressure so that a liquid-phase reaction can be realized.

In order to suppress catalyst deactivation, the reaction may also be carried out under coexistence of hydrogen.

The mode of the reaction may be either flow-type or batch-type. In the case of a flow-type reaction, the weight hourly space velocity (WHSV) is selected usually in a range of 0.01 to 50 h⁻¹, and preferably 0.1 to 30 h⁻¹; the smaller the WHSV, the higher the yield of adamantanes. When the reaction is carried out under a condition where the molar ratio of hydrogen to the tricyclic saturated hydrocarbon compound having 10 or more carbon atoms is selected usually in a range of 0 to 10, and preferably 0 to 5, the yield of adamantanes increases. On the other hand, in a batch-type reaction, the mass ratio of catalyst to raw material is selected usually in a range of 0.01 to 2, and preferably 0.05 to 1. In addition, the reaction time is usually about 1 to 50 hours.

### EXAMPLES

The present invention will be described further in detail with reference to Examples, but it should be understood that the invention is not limited in any way by these Examples.

In addition, definitions of terms are as follows:

(1) trimethylenenorbornane (TMN) conversion: (1—mass of TMN after reaction/mass of TMN before reaction)×100 (wt %);
(2) adamantane selectivity: [mass of adamantane produced/(mass of TMN before reaction—mass of TMN after reaction)]×100 (wt %);
(3) adamantane yield: (mass of adamantane produced/mass of TMN before reaction)×100 (wt %).

### Example 1

In a Teflon vessel, there were charged 113 g of purified water, 1.12 g of sodium aluminate, 0.38 g of sodium hydroxide, 7.07 g of hexamethyleneimine, and 8.56 g of fumed silica (Aldrich), and the mixture was stirred at room temperature for 0.5 hour to prepare a gel. The gel obtained was charged into an autoclave made of Teflon and, while stirring at 20 rpm by means of a hydrothermal synthesis equipment, was heated at 150° C. for 168 hours. The crystalline product obtained was collected by filtration, washed with water, and thereafter dried at 120° C. overnight. The dried crystalline product was calcined in an air atmosphere at 540° C. for 12 hours to obtain white powder.

To 4 g of the white powder obtained was added 400 g of a 1 mon aqueous solution of ammonium nitrate and after stirring the mixture under heating at 80° C. for 1 hour, the powder was collected by filtration and washed with water. By repeating this operation four times, ammonium ion exchange was carried out. After the ion exchange, the white powder was dried at 120° C. and, thereafter, calcined at 540° C. for 12 hours to obtain proton-type MCM-22 zeolite. This white powder was subjected to an X-ray diffraction pattern measurement by means of an X-ray diffractometer using copper K-alpha radiation. As a result, there was obtained the X-ray diffraction pattern shown in FIG. 1 and the white powder was confirmed to be MCM-22 zeolite.

In a reaction vessel made of stainless steel (SUS), 2 g of the catalyst obtained by the above-mentioned operations was charged and calcined under air flow at 300° C. for 3 hours. Thereafter, feeding of a 79 wt % solution of trimethylenenorbornane (TMN) in ethylcyclohexane was started and the reaction was carried out continuously under conditions of 300° C., reaction pressure of 6 MPa, and WHSV of 7 (based on TMN). The results after 50 hours from the start of raw material feeding are shown in Table 1.

### Example 2

Except that the reaction was conducted with WHSV of 3.5 h⁻¹ (based on TMN), the preparation and pretreatment of the catalyst and the reaction were carried out in the same manner as in Example 1. The results after 50 hours from the start of raw material feeding are shown in Table 1.

### Example 3

Except that the reaction was carried out with WHSV of 1.75 h⁻¹ (based on TMN), the preparation and pretreatment of the catalyst and the reaction were carried out in the same manner as in Example 1. The results after SO hours from the start of raw material feeding are shown in Table 1 and FIG. 4.

### Example 4

Except that the reaction was carried out with WHSV of 0.875 h⁻¹ (based on TMN), the preparation and pretreatment of the catalyst and the reaction were carried out in the same manner as in Example 1. The results after 50 hours from the start of raw material feeding are shown in Table 1.

### Example 5

Except that the reaction was carried out at a reaction temperature of 275° C., the preparation and pretreatment of the catalyst and the reaction were carried out in the same manner as in Example 1. The results after 50 hours from the start of raw material feeding are shown in Table 1.

### Example 6

Except that the reaction was carried out at a reaction temperature of 325° C., the preparation and pretreatment of the catalyst and the reaction were carried out in the same manner as in Example 1. The results after 50 hours from the start of raw material feeding are shown in Table 1.

### Example 7

In an autoclave of 100 ml volume, there were charged 1 g of a catalyst prepared in the same manner as in Example 1 and 5 g of the raw material TMN, and the reaction was carried out at 300° C. for 3 hours. The results are shown in Tables 1 and 2.

### Example 8

An aqueous solution containing 0.091 g (amount of Pt loading: 1.0 wt %) of Pt(NH₃)₄Cl₂.H₂O dissolved in 5 ml of pure water was prepared. In 50 g of pure water, 5 g of the MCM-22 prepared in Example 1 was suspended and the suspension was heated to 60° C. Under heating and stirring, the aqueous Pt(NH₃)₄Cl₂.H₂O solution was gradually added thereto. After addition of all of the aqueous Pt(NH₃)₄Cl₂.H₂O solution, the reaction mixture was stirred at 60° C. for 0.5 hour. The crystalline product obtained was collected by filtration, washed with water, and thereafter calcined in air at 300° C. for 3 hours to obtain MWW having Pt loaded thereon in an amount of 1.0 wt % (1.0% Pt/MWW). The catalyst, 2 g, obtained by the above-mentioned operations was filled in a reaction vessel made of SUS and reduced with hydrogen under normal pressure and hydrogen flow at 300° C. for 2 hours. Thereafter, feeding of a 79 wt % solution of trimethylenenorbornane (TMN) in ethylcyclohexane was started and the reaction was carried out continuously under the conditions of 300° C., reaction pressure of 6 MPa, WHSV of 7 h⁻¹ (based on TMN), and a molar ratio of hydrogen/TMN of 2.5. The results after 50 hours from the start of raw material feeding are shown in Table 1.

### Example 9

Except that the molar ratio of hydrogen/TMN was set at 1.5, the preparation of the catalyst and the reaction were carried out in the same manner as in Example 8. The results after 50 hours from the start of raw material feeding are shown in Table 1.

### Example 10

Except that the reaction temperature was set at 325° C., the preparation of the catalyst and the reaction were carried out in the same manner as in Example 8. The results after 50 hours from the start of raw material feeding are shown in Table 1.

### Example 11

Except that the amount of Pt loading was set at 0.2 wt %, the preparation of the catalyst and the reaction were carried out in the same manner as in Example 8. The results after 50 hours from the start of raw material feeding are shown in Table 1.

### Example 12

Except that the amount of Pt loading and the reaction temperature were set at 0.2 wt % and 325° C., respectively, the preparation of the catalyst and the reaction were carried out in the same manner as in Example 8. The results after 50 hours from the start of raw material feeding are shown in Table 1.

### Example 13

Except that the amount of Pt loading and WHSV were set at 2.0 wt % and 1.75 h⁻¹ (based on TMN), respectively, the preparation of the catalyst and the reaction were carried out in the same manner as in Example 8. The results after 50 hours from the start of raw material feeding are shown in Table 1 and FIG. 4.

### Example 14

Except that the amount of Pt loading was set at 1.5 wt %, the reaction was carried out in the same mariner as in Example 13. The results after 50 hours from the start of raw material feeding are shown in Table 1 and FIG. 4.

### Example 15

Except that WHSV was set at 1.75 (based on TMN), the preparation of the catalyst and the reaction were carried out in the same manner as in Example 8. The results after 50 hours from the start of raw material feeding are shown in Table 1 and FIG. 4.

### Example 16

Except that the amount of Pt loading was set at 0.5 wt %, the preparation of the catalyst and the reaction were carried out in the same manner as in Example 13. The results after 50 hours from the start of raw material feeding are shown in Table 1 and FIG. 4.

### Example 17

Except that the amount of Pt loading was set at 0.2 wt %, the preparation of the catalyst and the reaction were carried out in the same manner as in Example 13. The results after 50 hours from the start of raw material feeding are shown in Table 1 and FIG. 4.

### Example 18

Except that the amount of Pt loading was set at 0.1 wt %, the preparation of the catalyst and the reaction were carried out in the same manner as in Example 13. The results after 50 hours from the start of raw material feeding are shown in Table 1 and FIG. 4.

### Example 19

In a Teflon vessel, there were charged 113 g of purified water, 1.12 g of sodium aluminate, 0.38 g of sodium hydroxide, 7.07 g of hexamethyleneimine, and 8.56 g of fumed silica (Aldrich), and the mixture was stirred at room temperature for 0.5 hour to prepare a gel. The gel obtained was charged into an autoclave made of Teflon and, while stirring at 20 rpm by means of a hydrothermal synthesis equipment, was heated at 150° C. for 168 hours. The crystalline product obtained was collected by filtration, washed with water, and dried at 120° C. overnight. In a round-bottomed flask, there were added 3 g of the crystalline product obtained, 16.9 g of hexadecyltrimethylammonium bromide, and 74.5 g of a 10 wt % aqueous solution of tetrapropylammonium hydroxide, and the mixture was stirred under heating at 80° C. for 18 hours. Thereafter, the mixture was treated in an ultrasonic bath for 1 hour and its pH was adjusted to 2 or lower by adding several drops of concentrated hydrochloric acid. The white powder obtained was recovered by centrifugal separation. This white powder was dried at 120° C. and, thereafter, calcined in air at 540° C. for 12 hours to obtain ITQ-2 zeolite. This white powder was subjected to an X-ray diffraction pattern measurement by means of an X-ray diffractometer using copper K-alpha radiation. As a result, there was obtained the X-ray diffraction pattern shown in FIG. 2 and the white powder was confirmed to be ITQ-2 zeolite.

Thereafter, by carrying out transformation of the zeolite to proton-type zeolite and having Pt loaded thereon in the same manner as in Example 1 and Example 8, there was obtained ITQ-2 zeolite having Pt loaded thereon in an amount of 1.0 wt % (1.0 wt % Pt/ITQ-2). Using the catalyst obtained by the above-mentioned operations, the reaction was carried out in the same manner as in Example 8. The results after 50 hours from the start of raw material feeding are shown in Table 1,

### Example 20

In a Teflon vessel, there were charged 113 g of purified water, 1.12 g of sodium alurninate, 0.38 g of sodium hydroxide, 7.07 g of hexamethyleneimine, and 8.56 g of fumed silica (Aldrich), and the mixture was stirred at room temperature for 0.5 hour to prepare a gel. The gel obtained was charged into an autoclave made of Teflon and, while stirring at 20 rpm by means of a hydrothermal synthesis equipment, was heated at 150° C. for 168 hours. The crystalline product obtained was collected by filtration, washed with water, and thereafter dried at 120° C. overnight. In a flask were added 2 g of the crystalline product obtained, 2.26 g of hexadecyltrimethylammonium bromide, 2.44 g of a 40 wt % aqueous solution of tetrapropylammonium hydroxide, and 5.52 g of water, and the mixture was stirred under heating at 80° C. for 16 hours. Thereafter, by washing with water, filtration, and centrifugal separation, there was recovered a swollen intermediate having an MWW structure. After drying at 120° C., this was mixed with tetraethoxysilane (TEOS) in a mass ratio of 1 to 6 and the mixture was stirred in a nitrogen atmosphere under heating at 80° C. for 24 hours. Thereafter, 8 times mole of water relative to TEOS was added therein and the mixture was stirred for 5 hours under heating (90° C.). The powder was washed with water, collected by filtration, thereafter dried at 120° C., and calcined in air at 580° C. for 3 hours to obtain MCM-36 zeolite. This white powder was subjected to an X-ray diffraction pattern measurement by means of an X-ray diffractometer using copper K-alpha radiation. As a result, there was obtained the X-ray diffraction pattern shown in FIG. 3 and the white powder was confirmed to be MCM-36.

Thereafter, by carrying out transformation of the zeolite to proton-type one and having Pt loaded in the same manner as in Example 1 and Example 8, there was obtained MCM-36 zeolite having Pt loaded in an amount of 1.0 wt % (1.0 wt % Pt/MCM-36). Using the catalyst obtained by the above-mentioned operations, the reaction was carried out in the same manner as in Example 8. The results after 50 hours from the start of raw material feeding are shown in Table 1.

### Example 21

Except that the reaction was continued for 8 days from the start of raw material feeding in order to check the trend of catalyst deactivation, the catalyst preparation and the reaction were carried out in the same manner as in Example 3. The results are shown in FIG. 6.

From FIG. 6, it can be seen that in the MCM-22 zeolite catalyst system without loaded Pt, there is a slight trend of deactivation immediately after the start of raw material feeding but after 140 hours, there is almost no deactivation.

### Example 22

Except that the reaction was continued for 7 days after the start of raw material feeding in order to check the trend of catalyst deactivation, the catalyst preparation and the reaction were carried out in the same manner as in Example 15. The results are shown in FIG. 7.

From FIG. 7, it can be seen that in the MCM-22 zeolite system with Pt loaded in an amount of 1%, there is no trend of deactivation.

### Example 23

Except that perhydroacenaphthene was used as the raw material, the catalyst preparation and the reaction were carried out in the same manner as in Example 7. The results are shown in Table 2.

### Example 24

Except that perhydrofluorene was used as the raw material, the catalyst preparation and the reaction were carried out in the same manner as in Example 7. The results are shown in Table 2.

### Comparative Example 1

To 7000 g of pure water, 1275 g of Y-type zeolite having sodium ion at the cation sites was dispersed under stirring and the suspension was heated to 60° C. While stirring, 8 kg of an aqueous solution of mixed rare earth chloride (containing 890 g as RE₂O₃) was added thereto and stirring was continued for 2 hours. The powder was collected by filtration and, thereafter, washed with 15 kg of pure water. The washed material was dried at 110° C. for 12 hours and, thereafter, was calcined at 650° C. for 3 hours. The powder after calcination, 340 g, was suspended in 2 kg of warm water of 60° C. While stirring, hydrochloric acid was added until the pH became 5.01. To this slurry was added 2 kg of an aqueous solution of mixed rare earth chloride (containing 130.6 g as RE₂O₃) and the mixture was stirred at 60° C. for 2 hours. After the powder obtained was collected by filtration and washed with 4 kg of pure water, it was dried at 110° C. for 12 hours and, thereafter, calcined in air at 650° C. for 3 hours to obtain rare earth- loading Y-type zeolite (REY).

Using the catalyst obtained by the above-mentioned operations, the reaction was carried out in the same manner as in Example 1. The results after 50 hours from the start of raw material feeding are shown in Table 1.

### Comparative Example 2

Except that WHSV was set at 1.75⁻¹ (based on TMN), the catalyst preparation and the reaction were carried out in the same manner as in Comparative Example 1. The results after 50 hours from the start of raw material feeding are shown in Table 1 and FIG. 5.

### Comparative Example 3

REY, 400 g, obtained in Comparative Example 1 was suspended in 2 kg of pure water, 720 g of a 1.0% aqueous solution of platinum tetraammine chloride was added thereto, and the mixture was stirred at 30° C. for 2 hours. The powder was collected by filtration, washed, thereafter dried at 110° C. for 12 hours, and calcined in air at 350° C. for 3 hours to obtain REY having Pt loaded in an amount of 1.0 wt % (1.0 wt % Pt/REY).

Using the catalyst obtained by the above-mentioned operations, the reaction was carried out in the same manner as in Example 8. The results after 50 hours from the start of raw material feeding are shown in Table 1.

### Comparative Example 4

Except that the amount of Pt loading was set at 2.0 wt % and WHSV at 1.75⁻¹ (based on TMN), the preparation of the catalyst and the reaction were carried out in the same manner as in Comparative Example 3. The results after 50 hours from the start of raw material feeding are shown in Table 1 and FIG. 5.

### Comparative Example 5

Except that the amount of Pt loading was set at 1.5 wt %, the preparation of the catalyst and the reaction were carried out in the same manner as in Comparative Example 4. The results after 50 hours from the start of raw material feeding are shown in Table 1 and FIG. 5.

### Comparative Example 6

Except that the amount of Pt loading was set at 1.0 wt %, the preparation of the catalyst and the reaction were carried out in the same manner as in Comparative Example 4. The results after 50 hours from the start of raw material feeding are shown in Table 1 and FIG. 5.

### Comparative Example 7

Except that the amount of Pt loading was set at 0.5 wt %, the preparation of the catalyst and the reaction were carried out in the same manner as in Comparative Example 4. The results after 50 hours from the start of raw material feeding are shown in Table 1 and FIG. 5.

From FIGS. 4 and 5, it can be seen that, in the REY zeolite catalyst system, the conversion of TMN increases with increase in the amount of Pt loading and the yield of ADM also increases until the amount of Pt loading reaches 1.0 wt %. On the other hand, in the MCM-22 zeolite catalyst system, the yield of ADM increases with decrease in the amount of Pt loading and the value of the yield is higher compared to that in the case of the REY zeolite catalyst.

### Comparative Example 8

Except that the reaction was continued for 4 days from the start of raw material feeding in order to check the trend of catalyst deactivation, the preparation of the catalyst and the reaction were carried out in the same manner as in Comparative Example 2. In addition, the reaction was stopped after 4 days because the catalyst lost its activity. The results are shown in FIG. 8.

From FIG. 8, it can be seen that in the REY zeolite catalyst system without Pt loading, the catalyst deactivation faster.

### Comparative Example 9

Except that the reaction was continued for 7 days after the start of raw material feeding in order to check the trend of catalyst deactivation, the catalyst preparation and the reaction were carried out in the same manner as in Comparative Example 3. The results are shown in FIG. 9.

From FIG. 9, it can be seen that in the REY zeolite catalyst system, the catalyst shows trend of deactivation even when Pt is loaded in an amount of 1%.

| | | | Reaction | WHSV | | | | | | | | |
|---|---|---|---|---|---|---|---|---|---|---|---|---|
| | | | temper- | (based on | TMN | Catalyst | Reaction | | | TMN | ADM | ADM |
| | | Reaction | ature | TMN) | amount | amount | time | H₂/TMN | Pressure | conversion | selectivity | yield |
| | Catalyst | mode | (° C.) | (h⁻¹) | (g) | (g) | (h) | ratio | (MPa) | (wt %) | (wt %) | (wt %) |
| Example 1 | MCM-22 | Flow-type | 300 | 7 | — | — | — | — | 6 | 32.1 | 52.6 | 18.0 |
| Example 2 | MCM-22 | Flow-type | 300 | 3.5 | — | — | — | — | 6 | 54.4 | 56.0 | 30.4 |
| Example 3 | MCM-22 | Flow-type | 300 | 1.75 | — | — | — | — | 6 | 79.0 | 55.2 | 43.6 |
| Example 4 | MCM-22 | Flow-type | 300 | 0.875 | — | — | — | — | 6 | 86.5 | 54.3 | 47.0 |
| Example 5 | MCM-22 | Flow-type | 275 | 7 | — | — | — | — | 6 | 11.0 | 53.8 | 5.9 |
| Example 6 | MCM-22 | Flow-type | 325 | 7 | — | — | — | — | 6 | 50.2 | 55.8 | 28.0 |
| Example 7 | MCM-22 | Batch-type | 300 | — | 5 | 1 | 3 | — | 6 | 79.5 | 56.0 | 44.5 |
| Example 8 | 1.0 wt % Pt/MCM-22 | Flow-type | 300 | 7 | — | — | — | 2.5 | 6 | 36.1 | 23.0 | 8.3 |
| Example 9 | 1.0 wt % Pt/MCM-22 | Flow-type | 300 | 7 | — | — | — | 1.5 | 6 | 35.8 | 25.7 | 9.2 |
| Example 10 | 1.0 wt % Pt/MCM-22 | Flow-type | 325 | 7 | — | — | — | 2.5 | 6 | 58.5 | 25.0 | 14.6 |
| Example 11 | 0.2 wt % Pt/MCM-22 | Flow-type | 300 | 7 | — | — | — | 2.5 | 6 | 33.8 | 32.5 | 11.0 |
| Example 12 | 0.2 wt % Pt/MCM-22 | Flow-type | 325 | 7 | — | — | — | 2.5 | 6 | 51.8 | 34.0 | 17.6 |
| Example 13 | 2.0 wt % Pt/MCM-22 | Flow-type | 300 | 1.75 | — | — | — | 2.5 | 6 | 85.0 | 17.0 | 14.5 |
| Example 14 | 1.5 wt % Pt/MCM-22 | Flow-type | 300 | 1.75 | — | — | — | 2.5 | 6 | 83.5 | 17.6 | 14.7 |
| Example 15 | 1.0 wt % Pt/MCM-22 | Flow-type | 300 | 1.75 | — | — | — | 2.5 | 6 | 82.6 | 22.0 | 18.2 |
| Example 16 | 0.5 wt % Pt/MCM-22 | Flow-type | 300 | 1.75 | — | — | — | 2.5 | 6 | 81.0 | 25.4 | 20.6 |
| Example 17 | 0.2 wt % Pt/MCM-22 | Flow-type | 300 | 1.75 | — | — | — | 2.5 | 6 | 80.2 | 32.1 | 25.8 |
| Example 18 | 0.1 wt % Pt/MCM-22 | Flow-type | 300 | 1.75 | — | — | — | 2.5 | 6 | 80.0 | 42.5 | 34.0 |
| Example 19 | 1.0 wt % Pt/ITQ-2 | Flow-type | 300 | 7 | — | — | — | 2.5 | 6 | 42.0 | 18.5 | 7.8 |
| Example 20 | 1.0 wt % Pt/MCM-36 | Flow-type | 300 | 7 | — | — | — | 2.5 | 6 | 38.7 | 20.8 | 8.0 |
| Comparative | REY | Flow-type | 300 | 7 | — | — | — | — | 6 | 1.7 | 9.5 | 0.2 |
| example 1 | | | | | | | | | | | | |
| Comparative | REY | Flow-type | 300 | 1.75 | — | — | — | — | 6 | 3.6 | 9.5 | 0.3 |
| example 2 | | | | | | | | | | | | |
| Comparative | 1.0 wt % Pt/REY | Flow-type | 300 | 7 | — | — | — | 2.5 | 6 | 78.4 | 14.9 | 11.7 |
| example 3 | | | | | | | | | | | | |
| Comparative | 2.0 wt % Pt/REY | Flow-type | 300 | 1.75 | — | — | — | 2.5 | 6 | 100.0 | 11.0 | 11.0 |
| example 4 | | | | | | | | | | | | |
| Comparative | 1.5 wt % Pt/REY | Flow-type | 300 | 1.75 | — | — | — | 2.5 | 6 | 100.0 | 11.8 | 11.8 |
| example 5 | | | | | | | | | | | | |
| Comparative | 1.0 wt % Pt/REY | Flow-type | 300 | 1.75 | — | — | — | 2.5 | 6 | 98.2 | 13.1 | 12.9 |
| example 6 | | | | | | | | | | | | |
| Comparative | 0.5 wt % Pt/REY | Flow-type | 300 | 1.75 | — | — | — | 2.5 | 6 | 87.3 | 14.4 | 12.6 |
| example 7 | | | | | | | | | | | | |

TABLE 2

| | | | Reaction | | | | | | | |
|---|---|---|---|---|---|---|---|---|---|---|
| | | | temper- | Raw | Catalyst | Reaction | | | ADM | ADM |
| | | Reaction | ature | material | amount | time | Pressure | Conversion | selectivity | yield |
| | Catalyst | mode | (° C.) | (g) | (g) | (h) | (MPa) | (wt %) | (wt %) | (wt %) |
| Example 7 | MCM-22 | Batch-type | 300 | 5 | 1 | 3 | 6 | 79.5 | 56.0 | 44.5 |
| Example 23 | MCM-22 | Batch-type | 300 | 5 | 1 | 3 | 6 | 100.0 | 80.8 | 80.8 |
| Example 24 | MCM-22 | Batch-type | 300 | 5 | 1 | 3 | 6 | 100.0 | 37.5 | 37.5 |

### INDUSTRIAL APPLICABILITY

The present invention provides an industrially favorable production process whereby adamantanes can be produced in a high yield using a catalyst, which is used for the isomerization reaction of a tricyclic saturated hydrocarbon compound having 10 or more carbon atoms, does not require a complicated waste liquid treatment operation, and makes high-yield production of adamantanes possible.

1. A process for producing a compound having an adamantane structure, the process comprising:
exposing at least one reactant to at least one catalyst, to yield the compound having the adamantine structure,
wherein the at least one catalyst is selected from the group consisting of:
(a) a zeolite having MWW topology;
(b) a delaminated MWW zeolite; and
(c) an interlayer-expanded MWW zeolite prepared by treatment with a metal compound.

2. The process according to claim 1, wherein the (a) zeolite having MWW topology is present and is one selected from the group consisting of MCM-22, SSZ-25, ITQ-1, PSH-3, and ERB-1.

3. The process according to claim 1, wherein the (b) delaminated MWW zeolite is present and is ITQ-2.

4. The process according to claim 1, wherein the (c) inter-layer expanded MWW zeolite prepared by treatment with a metal compound is present and is MCM-36.

5. The process according to claim 1, wherein the exposing comprises isomerizing a tricyclic saturated hydrocarbon compound having 10 or more carbon atoms with the at least one catalyst.

6. The process according to claim 5, wherein the tricyclic saturated hydrocarbon compound is at least one selected from the group consisting of trimethylenenorbornane, dimethyltrimethylenenorbomane, perhydroacenaphthene, and perhydrofluorene.

7. The process according to claim 1, wherein the at least one catalyst is one having an active metal loaded on the catalyst.

8. The process according to claim 7, wherein the active metal is at least one metal belonging to any of Group 8 to Group 10 of the periodic table and Re.

9. The process according to claim 7, wherein the active metal is platinum.

10. The process according to claim 7, wherein an amount of the active metal loaded relative to the catalyst is 1 mass % or less.

11. The process according to claim 2, wherein the exposing comprises isomerizing a tricyclic saturated hydrocarbon compound having 10 or more carbon atoms with the at least one catalyst.

12. The process according to claim 3, wherein the exposing comprises isomerizing a tricyclic saturated hydrocarbon compound having 10 or more carbon atoms with the at least one catalyst.

13. The process according to claim 4, wherein the exposing comprises isomerizing a tricyclic saturated hydrocarbon compound having 10 or more carbon atoms with the at least one catalyst.

14. The process according to claim 11, wherein the tricyclic saturated hydrocarbon compound is at least one selected from the group consisting of trimethylenenorbornane, dimethyltrimethylenenorbornane, perhydroacenaphthene, and perhydrofluorene.

15. The process according to claim 12, wherein the tricyclic saturated hydrocarbon compound is at least one selected from the group consisting of trimethylenenorbornane, dimethyltrimethylenenorbornane, perhydroacenaphthene, and perhydrofluorene.

16. The process according to claim 13, wherein the tricyclic saturated hydrocarbon compound is at least one selected from the group consisting of trimethylenenorbornane, dimethyltrimethylenenorbornane, perhydroacenaphthene, and perhydrofluorene.

17. The process according to claim 2, wherein the at least one catalyst is one having an active metal loaded on the catalyst.

18. The process according to claim 3, wherein the at least one catalyst is one having an active metal loaded on the catalyst.

19. The process according to claim 4, wherein the at least one catalyst is one having an active metal loaded on the catalyst.

20. The process according to claim 5, wherein the at least one catalyst is one having an active metal loaded on the catalyst.
